(19) United States
(12) Patent Application Publication
(10) Pub. No.: US 2009/0137971 A1
(43) Pub. Date: May 28, 2009

(54) Phacoemulsification Needle Tips for Torsional Motion

(76) Inventor: Takayuki Akahoshi, Tokyo (JP)

Correspondence Address: LAW OFFICES OF JERRY A. SCHULMAN, 1S376 SUMMIT AVENUE, COURT C, OAKBROOK TERRACE, IL, 60181, US

(21) Appl. No.: 11/924,591

(22) Filed: Oct. 25, 2007

Related U.S. Application Data
(60) Provisional application No. 60/862,985, filed on Oct. 26, 2006.
(60) Provisional application No. 60/886,930, filed on Jan. 28, 2007.

(51) Int. Cl. A61M 5/32; B23P 11/00

(52) U.S. Cl. 604/272; 29/428

(57) ABSTRACT

Phacoemulsification needle tips are provided with emulsifying surfaces enhancing their capacity to emulsify eye tissue when used with phaco handpieces that vibrate such needles torsionally. In one version, prongs are formed on the tip proximate the tip mouth and are bent to be positioned to provide additional emulsifying surfaces in both needle rotational directions. In another version, additional emulsifying surfaces are formed on the interior of the tip or the needle.

### PRIORITY

This application claims priority from U.S. provisional patent application Ser. No. 60/862,985, filed Oct. 26, 2006 and entitled “Phacoemulsification Needle Tip for Torsional Motion”, and Ser. No. 60/886,930, filed Jan. 28, 2007 and entitled “Phacoemulsification Needle Tip with Interior Cutting Surfaces”, both of which are hereby incorporated herein by reference.

### FIELD OF THE INVENTION

This disclosure relates to surgical instruments and surgical techniques used in eye surgery and more particularly, to phacoemulsification needle tip designs for use with handpieces that produce torsional motion.

### BACKGROUND OF THE INVENTION

A common ophthalmological surgical technique is the removal of a diseased or injured lens from the eye. Earlier techniques used for the removal of the lens typically required a substantial incision to be made in the capsular bag in which the lens is encased. Such incisions were often on the order of 12 mm in length.

Later techniques focused on removing diseased lenses and inserting replacement artificial lenses through as small an incision as possible. For example, it is now a common technique to take an artificial intraocular lens (IOL), fold it and insert the folded lens through the incision, allowing the lens to unfold when it is properly positioned within the capsular bag. Similarly, efforts have been made to accomplish the removal of the diseased lens through an equally small incision.

One such removal technique is known as phacoemulsification. A typical phacoemulsification tool includes a handpiece to which is attached a hollow needle. Electrical energy is applied to vibrate the needle at ultrasonic frequencies in order to fragment the diseased lens into small enough particles to be aspirated from the eye through the hollow needle. Commonly, an infusion sleeve is mounted around the needle to supply irrigating liquids to the eye in order to aid in flushing and aspirating the lens particles.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small in incision as possible during such surgery is the minimization of leakage of liquid during and after surgery and the prevention of such a collapse.

Phacoemulsification needles and tips are well represented in the prior art. Needles and tips of varying configurations are well known. A particular shape for a tip or needle is often dictated by the type of handpiece with which the needle is to be used.

U.S. Pat. No. 5,725,495 (Strukel et al) teaches and describes a phacoemulsification handpiece, sleeve and tip illustrating a wide variety of tip configurations and needle cross-sectional configurations.

U.S. Pat. No. 6,007,555 (Devine) teaches and describes an ultrasonic needle for surgical emulsification. The needle and its tip are shown in both circular and oval configurations.

U.S. Pat. No. 6,605,054 (Rockley) teaches and describes a multiple bypass port phaco tip having multiple aspiration ports and a single discharge port to infuse liquid into the eye.

U.S. Pat. No. 5,879,356 (Geuder) teaches and describes a surgical instrument for crushing crystalline eye lenses by means of ultrasound and for removing lens debris by suction which demonstrates the use of a sleeve positioned concentric to the needle and having a pair of discharge ports formed thereon.

U.S. Pat. No. 5,645,530 (Boukhny) teaches and describes a phacoemulsification sleeve, one variation of which has a bellows portion attached to a discharge port ring which directs an annular flow of liquid around the needle and into the eye. The use of the bellows is intended to allow the sleeve to absorb spikes in liquid pressure during the operation.

Published U.S. Patent Application No. 2003/0004455 (Kadziauskas) teaches and describes a bi-manual phaco needle using separate emulsification and aspiration needles inserted into the eye simultaneously during surgery.

United States Patent Application Publication 2006/0217672 (Chon) teaches and describes a phacoemulsification tip that is swaged or crimped at its distal end. The tip is intended for use with a handpiece producing torsional motion and the crimping forms cutting edges at the distal end.

U.S. Pat. No. 6,077,285 (Boukhny) teaches and describes a torsional ultrasound handpiece configured to impart both longitudinal and torsional motion to a phacoemulsification needle.

U.S. Pat. No. 6,402,769 (Boukhny) is a continuation in part of the '285 patent and further particularizes the frequencies at which the crystals providing both the torsional and longitudinal motion are activated.

I have determined that improved results can be achieved using high-speed handpieces in both the longitudinal and torsional direction if the phacoemulsification tip is provided with a particular geometry. I have also determined that these improved results can be achieved using the straight phacoemulsification needle configuration, a configuration which is favored by a considerable number of doctors.

In accordance with these criteria, I have designed a series of tips that are specifically configured to enhance the emulsifying defect created by the handpiece in the torsional direction. In addition, the improved results are anticipated with the use of handpieces providing longitudinal motion.

In accordance with an example of the invention, a phacoemulsification needles is provided for use with a high-frequency torsional phacoemulsification handpiece with the needle having a series of prongs formed on the tip proximate the tip opening.

In a second example, the prongs extend over or into the tip opening.

In another example, the tip prongs are formed with curved outer surfaces.

In another example, the tip prongs have selected edges that are sharpened or beveled.

In another example, the prongs are bent or formed at different angles.

In accordance with another example of the invention, a phacoemulsification needle is provided for use with a high-frequency phacoemulsification handpiece with the needle tip having a series of individually-cutting or emulsifying surfaces formed on the interior of the tip and extending longitudinally within the tip.

In another example, the tip is formed from a planar sheet of titanium and rolled into a “spiral” configuration within the tip interior.

In another example, the tip opening is formed at different angles.

While the following describes an example or examples of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

### BRIEF DESCRIPTION OF THE DRAWINGS

These and further aspects of the present invention will be best understood by reference to the accompanying drawings wherein:

FIG. 1 is a drawing showing prior art oval and square-shaped tips;

FIG. 2 is a drawing showing several prior art needle cross-sectional configurations;

FIG. 3 is a partial lateral schematic sectional view of a tip having prongs formed at about a right angle to the tip wall;

FIG. 4 is a view along 4-4 of FIG. 3;

FIG. 5 is a partial schematic sectional view of a phaco tip having internal tip prongs that extend inward and curve downward;

FIG. 6 is a view along 6-6 of FIG. 5;

FIG. 7 is a partial schematic sectional view of a tip having prongs that curve downward and inward;

FIG. 8 is a view along 8-8 of FIG. 7;

FIG. 9 is a partial schematic sectional view of a tip having a square opening with prongs extending inward from the lip at about a 90 degree angle;

FIG. 10 is a view along FIG. 10-10 of FIG. 9;

FIG. 11 is a partial schematic sectional view of a square tip having prongs that extend inward and curve downward;

FIG. 12 is a view along 12-12 of FIG. 11;

FIG. 13 is a top plan view of a tip having prongs that curve outward and upward;

FIG. 14 is a partial schematic view taking along 14-14 of FIG. 13;

FIG. 15 is a top plan view of a tip showing 8 prongs curving upward and outward;

FIG. 16 is a top plan view of a tip having four foreshortened prongs;

FIG. 17 is a perspective view of the tip of 16;

FIG. 18 is a top plan view of a tip having two inwardly extending prongs and two inward and downwardly extending prongs;

FIG. 19 is a partial schematic sectional view of a tip having two downwardly and two inwardly extending prongs and two inwardly extending prongs;

FIG. 20 is a partial schematic sectional view of a tip having two outwardly extending and curving prongs and two inwardly and extending curving prongs;

FIG. 21 is a top view of a tip combining a pair of inwardly extending prongs, a pair of inward and downwardly extending prongs and a pair of outwardly curving prongs;

FIG. 22 is a partial perspective view of a straight phaco tip having a circular cross-section;

FIG. 23 is a partial perspective view of a tip of FIG. 22 showing areas along the tip edge of the tip marked for removal;

FIG. 24 shows the various areas in FIG. 23 having been removed;

FIG. 25 shows the remaining prongs folded over in the configuration of FIG. 4;

FIG. 26 is an end view of a tip constructed in accordance with a preferred embodiment of the present invention;

FIG. 27 is a partial lateral sectional view of the tip shown in FIG. 26 with a beveled configuration;

FIG. 28 is a schematic view of a sheet of metal from which a second embodiment of the invention is formed;

FIG. 29 is an end view of the tip formed from the sheet in FIG. 5; and

FIG. 30 is a lateral elevational view of the tip of FIG. 6 illustrating several preferred bevels.

### DETAILED DESCRIPTION OF THE INVENTION

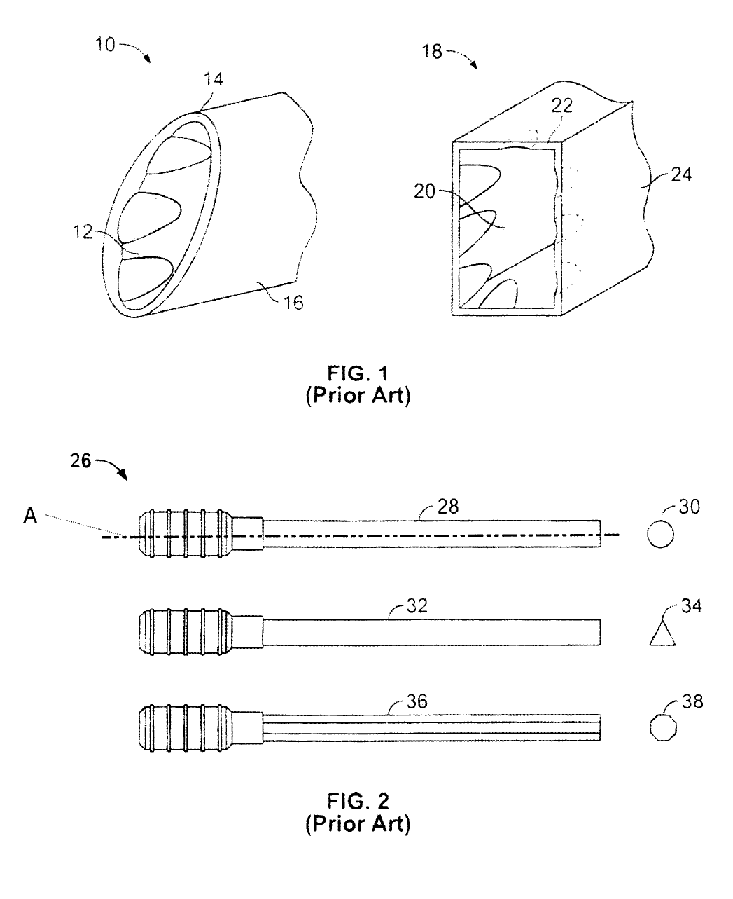

Referring now to FIG. 1, the numeral 10 indicates generally a prior art phacoemulsification needle tip as shown in U.S. Pat. No. 6,007,555. Needle 10 terminates in a mouth 12 defined by a lip 14 at the end of needle body 16, with lip 14 and needle body 16 formed as having an oval cross-section configuration.

Referring to FIG. 1, the numeral 18 indicates generally a prior art phacoemulsification needle tip from U.S. Pat. No. 6,007,555, having a mouth 20 defined by a lip 22 at the end of needle 24. The cross-sectional configuration of needle 18 and mouth 20 is a rectangle.

Referring now to FIG. 2, the numeral 26 identifies several prior art phacoemulsification needles as described in U.S. Pat. No. 5,725,495, with needle 28 having a circular cross-section as shown at 30, needle 32 having a triangular cross-section as shown at 34 and needle 36 having an octagonal cross-section as shown at 38.

Also shown in FIG. 2 is a central axis A extending down the length of needle 28. For the purposes of the following description, each phacoemulsification tip described herein is formed as a part of a needle shaft having a similar, centrally-located axis no matter the cross-sectional shape of the needle shaft.

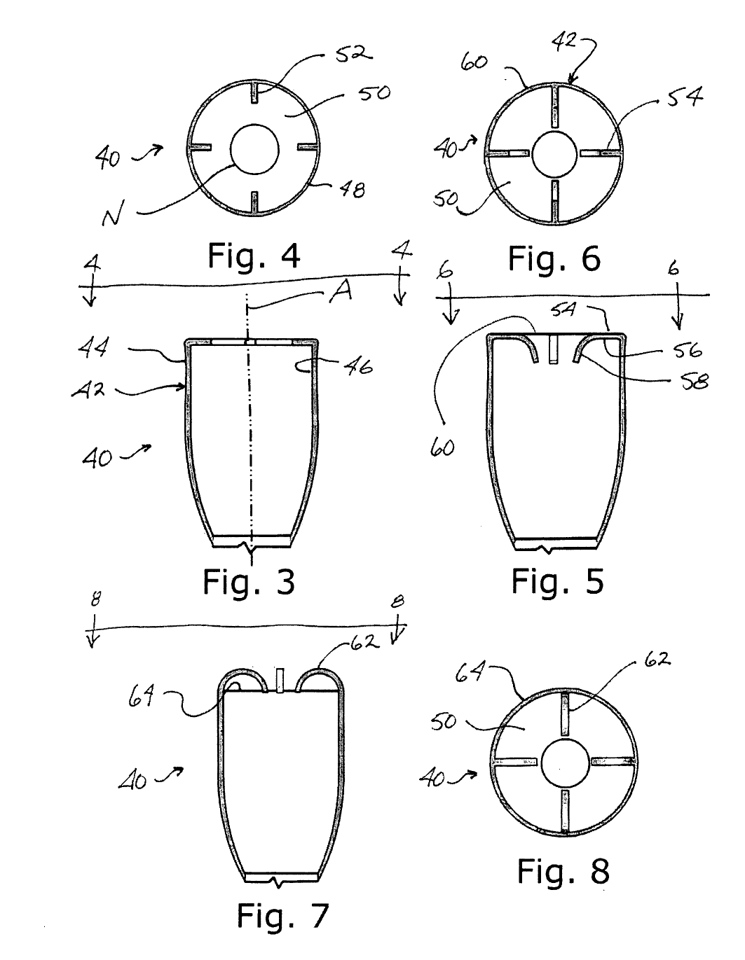

Referring now to FIG. 3, the numeral 40 indicates generally a phacoemulsification needle tip embodying certain aspects of the present invention. In this example, tip 40 is circular in cross section as seen in FIG. 4 and has an outer wall 42 having an outer surface 44 and an inner surface 46. As seen in FIG. 4, tip 40 also has a lip 48 extending about tip mouth 50. As described above, an axis A is shown in FIG. 3 and it should be understood that axis A is a reference point defined by the center of the phacoemulsification needle N upon which tip 40 is formed.

In the example shown in FIGS. 3 and 4, tip 40 has four internally extending tip projections or prongs 52 formed at about a 90 degree angle with outer wall 42 and extending into and partially across mouth 50. Preferably, prongs 52 are formed integrally with outer wall 42 at lip 48.

Referring now to FIGS. 5 and 6, tip 40 is shown having four prongs 54 which extend inwardly across and then curve downwardly into mouth 50. For the purposes of this description, the prong that extends at approximately a right angle to outer wall 42 will be referred to as inwardly projecting, a prong which extends into mouth 50 from outer wall 42 will be described as downwardly projecting, and a prong which extends at an angle greater than 90 degrees without outer wall 42 will be described as upwardly projecting.

In FIG. 5, each prong 54 is shown having a first, inwardly projecting segment 56 and a second, downwardly curving and projecting segment 58. Prongs 54 are preferably formed integrally with tip 40 at lip 60.

Referring now to FIGS. 7 and 8, tip 40 is shown having four identically formed prongs 62 in arcuate shapes. As seen in FIG. 7, prong 62 extends integrally with and from outer wall 42 to curve upward and then downward toward or into mouth 50. Lip 64 extends around the periphery of mouth 50 intermediate prongs 62.

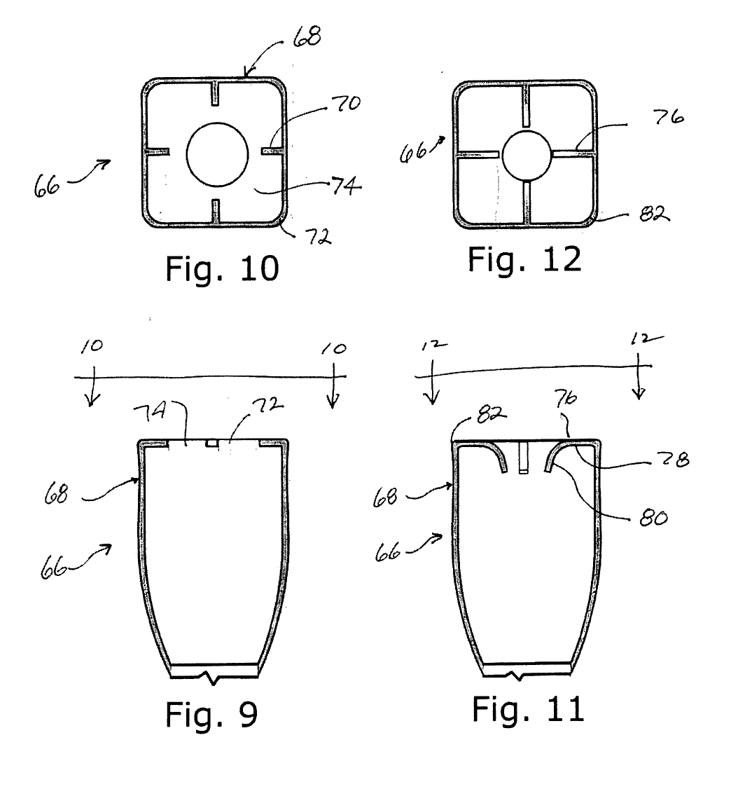

Referring now to FIGS. 9 and 10, a square tip 66 has an outer wall 68 and four identically formed and inwardly extending prongs 70. As described above, prong 70 are preferably formed integrally with outer wall 68 at lip 72 which, with outer wall 68 defines mouth 74.

Referring now to FIGS. 11 and 12, square tip 66 is shown having four identically formed prongs 76 with each prong 76 having a first inwardly extending segment 78 and a second downwardly curving segment 80. Prongs 76 are preferably formed integrally with outer wall 68 at lip 82.

The foregoing examples have all been shown with four prongs. It should be understood and appreciated that any number of prongs in any desired spacing may be utilized as desired. For example, 3, 6 or 8 prongs may also be used. The selection of the number of prongs will reside more with the predicted or observed efficiency of the tip when used with a torsional handpiece. For example, it may be more efficacious to have the prongs equidistantly spaced no matter how many prongs are used to balance the tip.

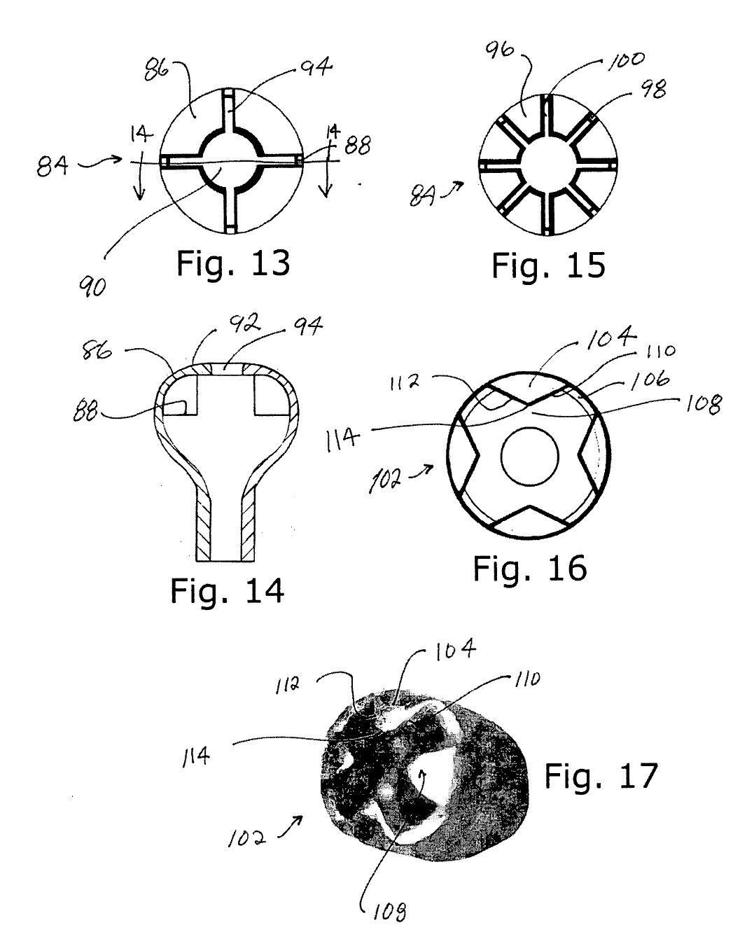

Referring to FIG. 13, a top plan view is shown of a circular tip 84 having four curved and upwardly extending prongs 86. The prongs are formed integrally with tip 84 at lip 88 and extend upwardly from lip 88 curving inwardly to extend over mouth 90.

As seen in FIG. 14, each prong 86 has an arcuate outer surface 92. As seen in FIGS. 13 and 14, each prong 86 is separated from its adjacent neighbor by a groove or bight 94 and extends upward from lip 88.

Referring now to FIG. 15, a second example of tip 84 is shown with 8 prongs 96 formed thereon. Each prong extends upwardly from and is integral with lip 98 and each is separated by a groove or bight 100.

Referring now to FIG. 16, a circular tip 102 has formed thereon four curved and upwardly extending prongs 104 formed integrally with and extending upwardly from lip 106. Prongs 104 are foreshortened when compared to prongs 86, 96, leaving a wider access to mouth 108. Each prong 104 in FIG. 16 has a first edge 110 and a second edge 112 meeting at an apex 114. If desired, apex 114 may be rounded. Such a configuration is shown in perspective in FIG. 17 showing in greater detail the geometry of each prong 104.

It is another feature of the present invention to provide prongs such as those hereinabove described in not only varying sizes and shapes but, in some instances, with edges that are honed, beveled or otherwise sharpened.

For example, referring to FIG. 17, edges 110 and 112 together with apex 114 may be sharpened or beveled. In FIG. 13, edges 166, 168, 170 of prong 86 may also be sharpened, if desired. The decision to hone or sharpen a selected edge of a selected prong again depends upon the use to which the tip is to be put, the type of handpiece, the feel of the tip so constructed and the preference of the surgeon

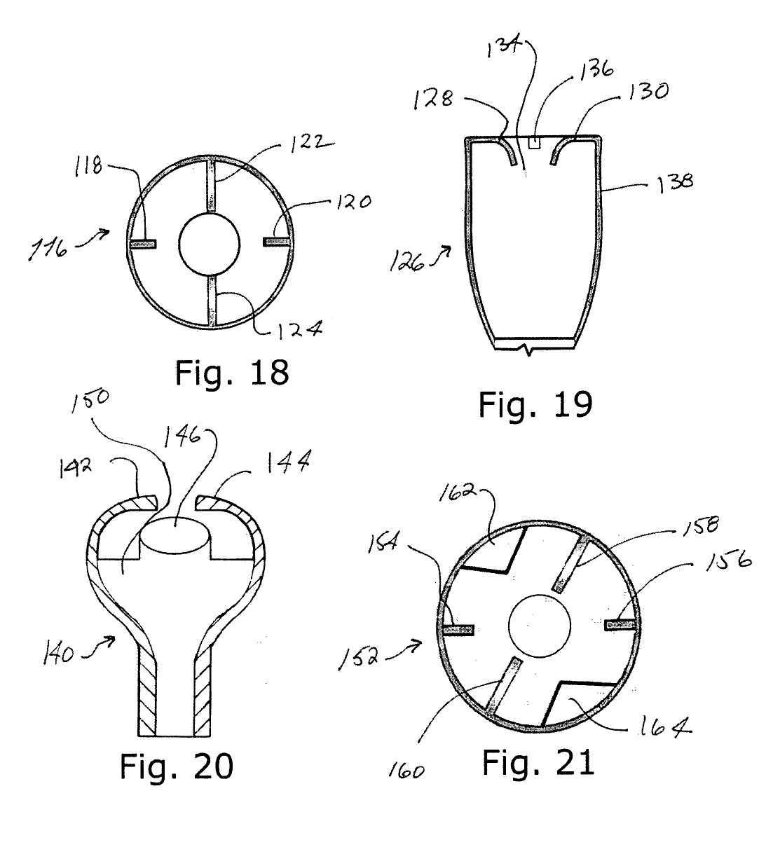

Referring now to FIG. 18, the numeral 116 identifies a tip having four prongs formed thereon with prongs 118, 120 substantially identical to prongs 52 as shown in FIG. 4 and with prongs 122, 124 substantially identical to prongs 54 as shown in FIGS. 5 and 6. In this manner, one may take advantage of the different cutting characteristics of each such prong. The prongs are shown in FIG. 18 as equidistantly spaced with identical prongs positioned directly opposite one another. It is also possible to intermix these prongs again depending upon the preferences of the user, and with consideration paid to the balance, feel and operation of the handpiece being used.

Referring now to FIG. 19 another example of a circular tip 126 is shown having prongs 128, 130 formed thereon substantially identical to prongs 54 shown in FIGS. 5 and 6, but extending downwardly into mouth 134. A second pair of prongs 136 and 138 (not shown) are formed on tip 126 again diametrically opposed and equidistantly spaced with prongs 136, 138 substantially identical to prong 52 and extending at approximately a right angle to wall 138 of tip 126.

Referring now to FIG. 20, a tip 140 is shown in cross-section having a pair of curved outwardly extending prongs 142, 144 formed thereon and a second pair of curved prongs 146, 148 (not shown) curved inwardly and downwardly into mouth 150.

Referring now to FIG. 21, the number 152 identifies a tip combining a first pair of prongs 154, 156 constructed to extend inwardly as shown with respect to prong 52 in FIGS. 3 and 4, a second set of prongs 158, 160 extending inwardly and downwardly as shown at prong 54 in FIGS. 5 and 6, and a third pair of prongs 162, 164 extending and curving upwardly and inwardly as seen in at prong 104 in FIG. 17.

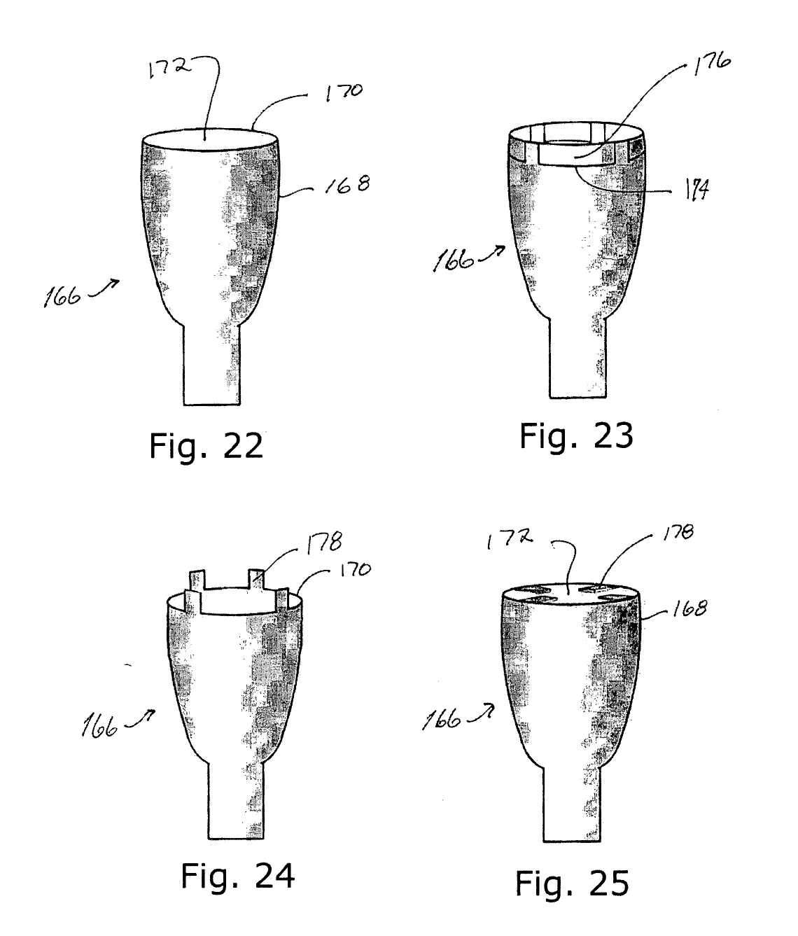

Referring now to FIG. 22 the numeral 166 identifies a round, straight phacoemulsification tip prior to the formation of prongs as described above. Tip 166 has an outer wall 168 and a lip 170 proximate mouth 172.

Referring to FIG. 23, tip 166 is shown with lines 174 setting off wall segments 176 to be cut or otherwise removed as a step toward making a tip such as that depicted in FIGS. 3 and 4. In this example, four such segments 176 are so marked.

FIG. 24 illustrates the configuration of tip 166 after segments 176 have been severed and removed, leaving upstanding tabs or prongs 178 extending upward from and integral with tip 166 at lip 170.

In FIG. 25, tip 166 is shown with prongs 178 bent inward to an angle of about 90° with wall 168 at lip 170 to overlap mouth 172. Tip 166 is now complete.

It should be understood that the process described in connection with FIGS. 22-25 can also be used to form prongs with configurations as described hereinabove.

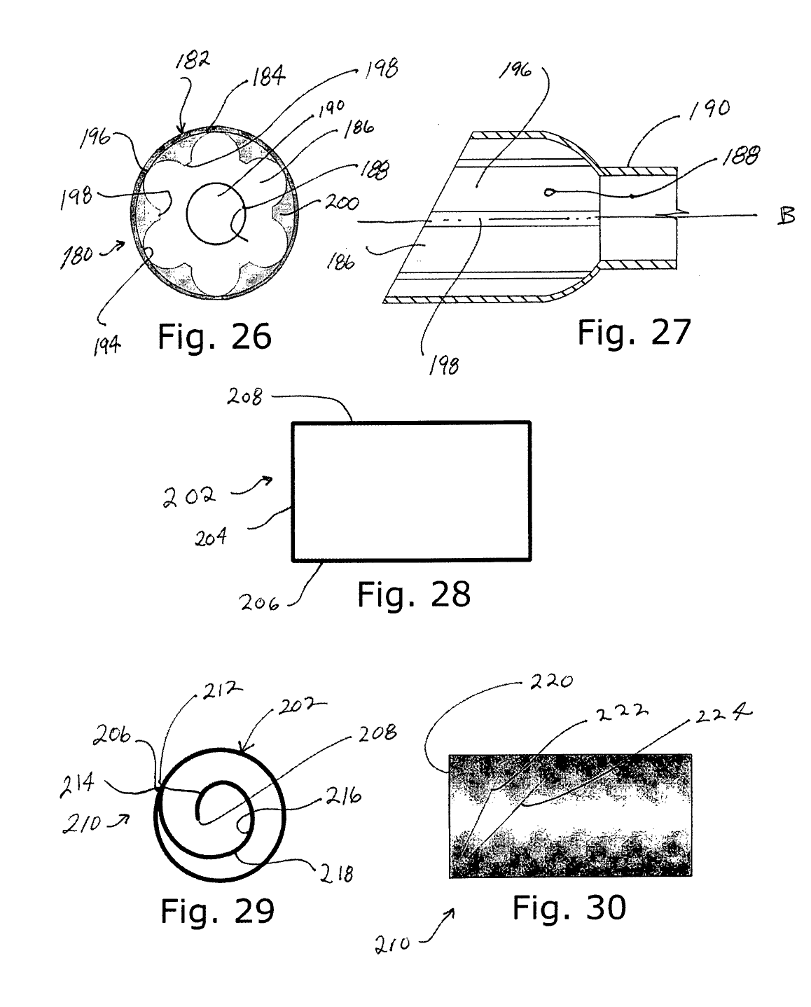

Referring now to FIG. 26, the numeral 180 indicates generally a phacoemulsification needle tip embodying certain aspects of the present invention. In this example, tip 180 is circular in cross section and has an outer wall 182 terminating at one end in a lip 184 which defines a tip opening 186. A central cavity 188 extends from tip opening 186 in a rearward or longitudinal direction as seen also in FIG. 27.

As seen in FIGS. 26 and 27, tip 180 terminates at a hollow needle body 190 having a central passageway 192, corresponding to the needle passageway N described earlier, which communicates with cavity 188 and tip opening 186 to form a flow path for fluid and emulsified tissue removed from a patient's eye.

As seen in FIG. 26 the interior surface 194 of wall 182 is formed in a “scalloped” cross-sectional configuration with a series of semi-circular sections 196 meeting at flattened peaks 198 to form a series of longitudinally-extending flattened ridges 200. For the purposes of this description, a “scallop” is understood to be that portion of interior surface 194 extending upward to and downward from a flattened peak 198.

As seen laterally in FIG. 27, each section 196 and each peak 198 extend longitudinally along the interior of tip 180 parallel to central axis B of tip 180, forming a series of longitudinally-extending ridges 200 extending into cavity 188.

The surfaces of semi-circular sections 196 form emulsifying or “cutting” surfaces when tip 180 is rotated in a torsional direction about axis B and will emulsify tissue along the interior of tip 180.

Referring now to FIG. 28, the numeral 202 identifies a generally rectangular sheet of material thin enough and stiff enough to be suitable for the construction of a phacoemulsification tip. One example of such a material is titanium. Sheet 202 has a lateral edge 204, a first longitudinal edge 206 and a second longitudinal edge 208.

As seen in FIG. 29, a phacoemulsification needle 210 is formed by rolling sheet 202 into a right cylindrical spiral configuration, with first longitudinal edge 206 attached to sheet 202 at seam 214 and with second longitudinal edge 208 remaining free and extending longitudinally along the length of tip 210. The spiral formed by sheet 202 thus has a “tail” 212 that terminates at second longitudinal edge 208 at one end and is attached to tip 210 at seam 212 at the other end. Tail 212 has an “inner” surface 216 and an “outer” surface 218. When tip 210 is used with a torsional handpiece, surfaces 216, 218 form “cutting” or emulsifying surfaces when tip 210 is rotated clockwise and counterclockwise as viewed in FIG. 29.

Referring now to FIG. 30, tip 210 is shown in a lateral elevation. Tip 210 is formed in a straight configuration when lip 220 is unbeveled. When lip 220 is formed along cut line 222 tip 210 has a 30° bevel, and when formed along cut line 224 has a 45° bevel.

It is expected that others skilled in the art will determine that other manufacturing processes and techniques can also be used to form the tip examples described and claimed herein.

I claim:

1. A phacoemulsification needle for use with a phaco handpiece, said needle comprising:
a hollow shaft having a proximal end, a distal end and a central axis,
said proximal end adapted to be attached to said handpiece;
a needle tip formed at said distal end of said shaft,
said tip having a side wall terminating in a lip;
said lip defining a tip mouth;
means formed on said tip comprising an emulsifying surface when said tip is rotated torsionally about said axis,
said surface means formed at said lip and extending into said mouth toward said axis.

2. The apparatus as recited in claim 1 wherein said surface means comprises at least one prong.

3. The apparatus as recited in claim 2 wherein said at least one prong has at least a first edge surface,
said edge surface comprising an emulsifying surface when said needle is rotated torsionally in a first direction about said axis.

4. The apparatus as recited in claim 2 wherein each said at least one prong has at least a first edge surface and a second edge surface,
said first edge surface comprising a first emulsifying surface when said needle is rotated torsionally in a first direction about said axis, and
said second edge surface comprising a second emulsifying surface when said needle is rotated torsionally in a second direction about said axis.

5. The apparatus as recited in claim 4 wherein at least one said edge surface has a bevel formed thereon.

6. The apparatus as recited in claim 2 wherein at least one said prong is formed at said lip and extends at about a 90° angle to said tip side wall.

7. The apparatus as recited in claim 2 wherein at least one said prong is formed at said lip and comprises a first prong segment that extends at about a 90° angle to said tip side wall and a second prong segment extending at an angle from said first prong segment and into said mouth.

8. The apparatus as recited in claim 2 wherein at least one said prong is formed at said lip and comprises a curved prong segment that extends from said lip toward said axis.

9. The apparatus as recited in claim 1 wherein said side wall has a square cross-section, forming a square tip.

10. The apparatus as recited in claim 9 wherein at least one said prong is formed at the midpoint of one side of said square tip.

11. The apparatus as recited in claim 1 wherein at least one said prong is substantially triangular in shape.

12. The apparatus as recited in claim 2 wherein said surface means comprises four said prongs.

13. A phacoemulsification needle for use with a phaco handpiece, said needle comprising:
a hollow shaft having a proximal end, a distal end and a central axis,
said proximal end adapted to be attached to said handpiece;
a needle tip formed at said distal end of said shaft,
said tip having an outer side wall and an inner side wall, said sidewalls terminating in and forming a lip;
said lip defining a tip mouth;
means formed on said tip comprising an emulsifying surface when said tip is rotated torsionally about said axis,
said surface means formed extending from said lip into said mouth in the direction of said axis.

14. The apparatus as recited in claim 13 wherein said surface means comprises at least one scallop formed longitudinally along said inner side wall.

15. The apparatus as recited in claim 14 wherein each said at least one scallop comprises at least a first edge surface,
said edge surface comprising an emulsifying surface when said needle is rotated torsionally in a first direction about said axis.

16. The apparatus as described in claim 14 wherein each said at least one scallop has at least a first edge surface and a second edge surface,
said first edge surface comprising a first emulsifying surface when said needle is rotated torsionally in a first direction about said axis, and
said second edge surface comprising a second emulsifying surface when said needle is rotated torsionally in a second direction about said axis.

17. The apparatus as recited in claim 16 wherein said surface means comprises six said scallops.

18. A phacoemulsification needle for use with a phaco handpiece, said needle comprising:
a planar plate of material suitable for the manufacture of said phacoemulsification needle,
said plate having at least first and second opposed edges, a third edge extending from said first edge to said second edge and a plate surface extending between said edges,
said first edge rolled toward said second edge about a central axis to form said plate into a spiral shape,
said second edge fastened to said surface at a seam thereby forming a tube with the portion of said plate extending from said seam to said first edge held in said spiral shape,
said third edge forming a needle mouth.

19. The apparatus as recited in claim 18 wherein said needle mouth is formed at an angle with respect to said axis.

20. The apparatus as recited in claim 19 wherein said plate is titanium.

21. A method for making a phacoemulsification needle for use with a phaco handpiece, said needle of the type having a hollow shaft having a proximal end, a distal end and a central axis, said proximal end adapted to be attached to said handpiece, a needle tip formed at said distal end of said shaft, said tip having a side wall terminating in a lip, said lip defining a tip mouth, said method comprising the steps of:
severing portions of said side wall proximate said lip;
removing said side wall portions to form at least one prong extending from said lip; and
bending said prong toward said axis to position said prong at said tip mouth.

22. The method as recited in claim 21 including the step of forming a plurality of said prongs.

23. The method as recited in claim 22 including the step of forming said at least one prong to extend at an angle of about 90° to said side wall.

24. The method as recited in claim 22 including the step of forming said at least one prong to have a curved prong segment that extends from said lip toward said axis.

25. The method as recited in claim 22 including the steps of forming said at least one prong with a first edge surface and a second edge surface,
said first edge surface comprising a first emulsifying surface when said needle is rotated torsionally in a first direction about said axis, and
said second edge surface comprising a second emulsifying surface when said needle is rotated torsionally in a second direction about said axis.
